(19) United States
(12) Patent Application Publication
(10) Pub. No.: US 2013/0261654 A1
(43) Pub. Date: Oct. 3, 2013

(54) MATERIALS AND METHODS FOR IMPROVED INTRAGASTRIC BALLOON DEVICES

(71) Applicants: Outhit Bouasaysy, Corona, CA (US); Mark Ashby, Laguna Niguel, CA (US)

(72) Inventors: Outhit Bouasaysy, Corona, CA (US); Mark Ashby, Laguna Niguel, CA (US)

(73) Assignee: RESHAPE MEDICAL, INC., San Clemente, CA (US)

(21) Appl. No.: 13/877,644

(22) PCT Filed: Oct. 7, 2011
(86) PCT No.: PCT/US11/55373

§ 371 Date: Jun. 14, 2013

Related U.S. Application Data
(60) Provisional application No. 61/390,996, filed on Oct. 7, 2010.

(51) Int. Cl. A61F 5/00; A61M 25/10

(52) U.S. Cl. CPC A61F 5/003; A61M 25/1029
USPC 606/192; 264/299; 264/334

(57) ABSTRACT

An inflatable balloon of an intragastric device comprises a substantially homogonous lend of polydiphenylsiloxane and polydimethylsiloxane. The substantially homogenous blend improves elongation percentage, tear strength, permeability, and modulus as compared to its constituent parts taken alone.

### CROSS-REFERENCE TO RELATED APPLICATION(S)

This application claims the benefit of U.S. Provisional Application No. 61/390,996, filed Oct. 7, 2010, entitled “MATERIALS AND METHODS FOR IMPROVED INTRAGASTRIC BALLOON DEVICES,” which is incorporated herein by reference in its entirety.

This application also incorporates herein by reference the following patent publications and applications in their entireties: U.S. Patent Publication No. 2007/0100367, published May 3, 2007; U.S. Patent Publication No. 2007/0100368, published May 3, 2007; U.S. Patent Publication No. 2007/0100369, published May 3, 2007; U.S. Patent Publication No. 2007/0149994, published Jun. 28, 2007; U.S. Patent Publication No. 2008/0243071, published Oct. 2, 2008; U.S. Patent Publication No. 2008/0319471, published Dec. 25, 2008; U.S. Patent Publication No. 2005/0159769, published Jul. 21, 2005; U.S. Patent Publication No. 2009/0048624, published Feb. 19, 2009; WIPO Publication No. WO 2007/053556, published Oct. 5, 2007; WIPO Publication No. WO 2007/053707, published Oct. 5, 2007; WIPO Publication No. WO 2007/053706, published Oct. 5, 2007; and WIPO Publication No. WO 2007/075810, published May 7, 2007.

### TECHNICAL FIELD

This disclosure relates to implantable, expandable gastric devices. In particular, this disclosure relates to improved structures of balloons and methods of producing the same.

### SUMMARY

An intragastric device, comprising, in combination: a molded inflatable balloon, comprising a substantially homogonous blend of polydiphenylsiloxane and polydimethylsiloxane material.

An amount of the polydimethylsiloxane of the inflatable balloon may exceed an amount of the polydiphenylsiloxane of the inflatable balloon, by weight. The polydiphenylsiloxane may be about 15% of the inflatable balloon, by weight. The inflatable balloon may be mostly of the polydimethylsiloxane, by weight.

The inflatable balloon further may comprise a therapeutically active material. The therapeutically active material may be an anti-microbial additive.

The substantially homogonous blend may be of a greater elongation percentage than the polydiphenylsiloxane alone. The substantially homogonous blend may have a greater tear strength than the polydiphenylsiloxane alone or the polydimethylsiloxane alone. The substantially homogonous blend may have a lower permeability than the polydimethylsiloxane or a combination blend with polydiphenylsiloxane material. The substantially homogonous blend may have a greater modulus than the polydimethylsiloxane alone.

The intragastric device may further comprise a shaft. The inflatable balloon may further comprise two cuffs configured to receive the shaft extended through the inflatable balloon.

A method of producing an inflatable balloon, comprising, in combination: providing a mold and mandrel; providing a substantially homogonous blend of polydiphenylsiloxane and polydimethylsiloxane material within the mold and around the mandrel, whereby the balloon is defined by the space between the mold and the mandrel, the balloon having a cuff at an end thereof and a middle diameter corresponding to a middle diameter of the mandrel; removing the mold from about the balloon and the mandrel from within the balloon through the cuff.

The middle diameter of the mandrel may be between about 400% and about 600% larger than a diameter of the cuff. The expansion capabilities of the substantially homogonous blend may facilitate expansion of the cuff to allow exit of the mandrel.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. Moreover, in the drawings, like reference numerals designate corresponding parts throughout the several views.

FIG. 1 shows a perspective view of a balloon;

FIG. 2 shows a perspective view of an intragastric device;

FIG. 3 shows a side view of an intragastric device;

FIG. 4 shows a top view of an intragastric device;

FIG. 5 shows a sectional view of a balloon with a magnified view;

FIG. 6 shows a sectional view of a balloon with a magnified view; and

FIG. 7 shows a sectional view of a balloon with a magnified view.

### DETAILED DESCRIPTION

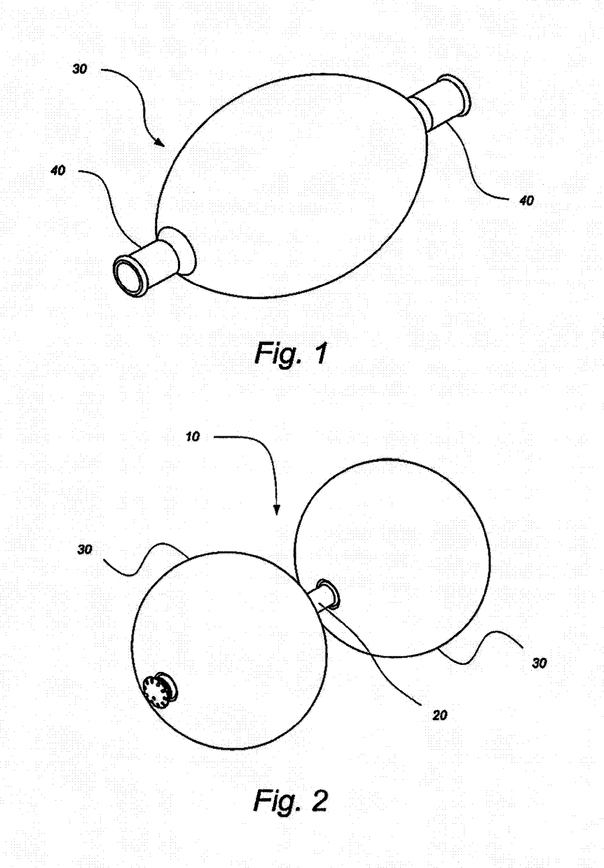

Specific details of several embodiments of the technology are described below with reference to FIGS. 1-7. Although many of the embodiments are described below with respect to devices, systems, and methods associated with intragrastric space fillers, other applications and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-7.

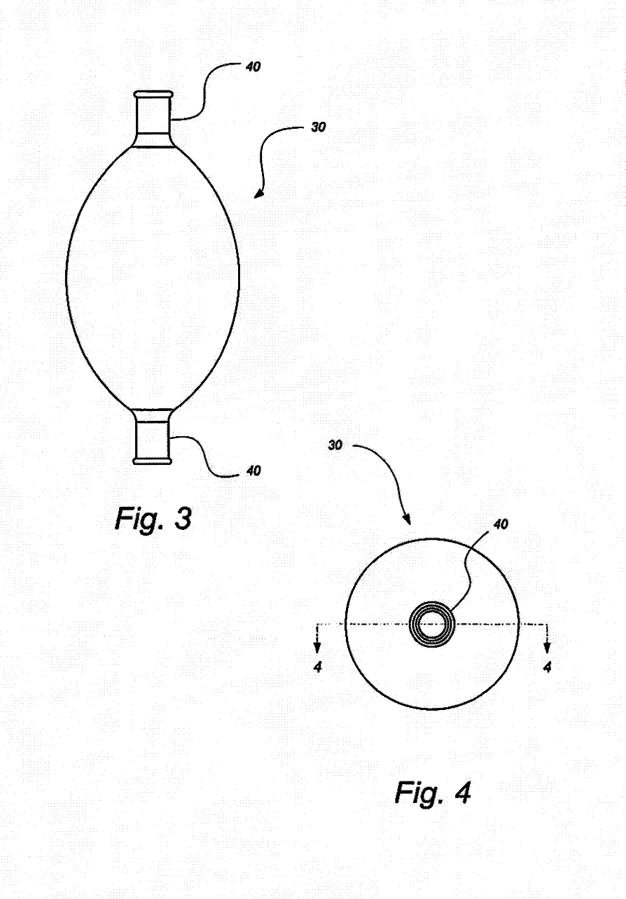

According to embodiments, and as shown in FIG. 1, balloon 30 is any expandable, space filling component. Balloon 30 may have any variety of geometries and shapes. As shown in FIGS. 1 and 3, balloon 30 may have at least one cuff 40 for interfacing with other components, such as shaft 20 extending through balloon 30. Balloon 30 may be an open or closed balloon. Balloon 30 may have an inner surface and outer surface.

According to embodiments, and as shown in FIG. 2, at least one balloon 30 may be a component of an intragastric device 10. As further shown in FIG. 2, a plurality of balloons 30 may be joined by a common shaft 20 extended and connecting the plurality of balloons 30.

According to embodiments, intragastric device 10 with at least one balloon 30 may be configured for use as an implantable device within a gastric cavity. Where implant is temporary, intragastric device 10 must be explanted after some period of time. Durability and longevity of intragastric device 10 may be defined, at least in part, by characteristics of balloon 30. Balloon 30 may be subjected to harsh gastric environments for an extended amount of time. Accordingly, the materials and manufacturing methods of the materials are key contributors as to balloon integrity and longevity.

According to embodiments, balloon 30 may be of at least one silicone-containing material. Two examples of such materials are polydimethylsiloxane (PDMS) and polydiphenylsiloxane (PDPS). PDMS may be represented as [SiO(CH₃)₂]ₙ or graphically as follows:

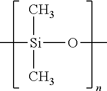

PDPS may be represented as [SiO(C₆H₅)₂]ₙ or graphically as follows:

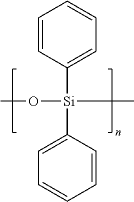

According to embodiments, at least one of PDMS and PDPS may form the base material of at least a portion of balloon 30. Other materials, structures, or compounds may be mixed or cross-linked with the based material.

According to embodiments, a plasma etching or coating may be provided to at least a portion of balloon 30. Plasma etching may involve a high-speed stream of glow discharge (plasma) of an appropriate gas mixture being shot (in pulses) at a sample. The plasma source can be either charged (ions) or neutral (atoms and radicals). During the process, the plasma will generate volatile etch products at room temperature from the chemical reactions between the elements of the material etched and the reactive species generated by the plasma. The atoms of the shot element embed themselves at or just below the surface of the target, thus modifying the physical properties of balloon 30. Etching may facilitate better adherence between layers of balloon 30.

According to embodiments, various coatings (e.g., hydrophilic) may be applied to at least a portion of balloon 30. For example, a hydrophilic coating may be provided where two surfaces of balloon 30 resist flow of fluid there through.

The respective chemical structures of PDMS and PDPS confer relatively disparate characteristics, which may be summarized as follows:

TABLE 1

| | Polydimethylsiloxane | Polydiphenylsiloxane |
|---|---|---|
| Manufacturing method: | Molding, extrusion | Dipping |
| Curing: | Heat | Heat |
| Consistency: | More dimensional | Less dimensional |
| | (moldable) | (dipping) |
| Elasticity: | Higher | Lower |
| Tensile Strength: | Lower | Higher |
| Acid resistance: | Acceptable | Increased |
| Permeability: | Higher | Lower |
| Molecular density: | Lower | Higher |
| Polymer Chain Length | Standard | Longer |

In various applications and based on different aspects, each of PDMS and PDPS may be seen as providing certain advantages and disadvantages.

According to embodiments, the more dimensional consistency and higher elasticity of PDMS materials and PDMS blends provide balloon 30 facilitate molding and extrusion processes. In particular, molding processes involving mandrels are better served by materials with higher elasticity due to the stresses involved in the mandrel removal process. For example, balloon 30 may be defined by a mandrel that has a larger diameter at a central portion than a diameter at one or both ends (corresponding to cuffs 40). By further example, a mandrel may have a middle diameter that is up to 600% larger than its end diameters. Balloon 30 results in a corresponding middle diameter and opening through cuff 40. Balloon 30 must provide enough elasticity to have the mandrel with middle diameter removed through cuff 40 without damaging cuff 40. As shown in Table 1, PDMS materials generally provides greater elasticity capabilities than PDPS materials.

According to embodiments, the expansion ratio of balloon 30 from an uninflated state (i.e., before and during implant) to an inflated state (i.e., after implant) may be significant. For example, balloon 30 may have an outer diameter of about 1.9″ in an uninflated state and about 4″ in an inflated state (over 200% expansion). By further example, balloon 30 may have a volume of about 80 cc in an uninflated state and about 450-500 cc in an inflated state (over 600% expansion). These factors are well-served by PDMS materials and PDMS based blends.

According to embodiments, higher acid resistance, a longer polymer chain (to resist degradation), double bonds (to resist degradation), increased hydrophobicity and less permeability (to reduce ingress/egress of materials) of PDPS materials and PDPS blends would better limit the ingress or egress of materials across the walls of balloon 30 and support longevity of balloon 30. Generally, PDPS, as compared to PDMS, is less able to provide consistent wall thicknesses and is less capable of generating molding friendly features because PDPS based materials generally are higher consistency in raw form (thicker & dense) and do not pump well in liquid injection molding (LIM) presses.

According to embodiments, balloon 30 may have a multiple-material composition. A plurality of disparate materials and material blends may be provide in layers forming walls of balloon 30. For example, a wall of balloon 30 may have a core 50 with at least one coating on an inner or outer surface thereof.

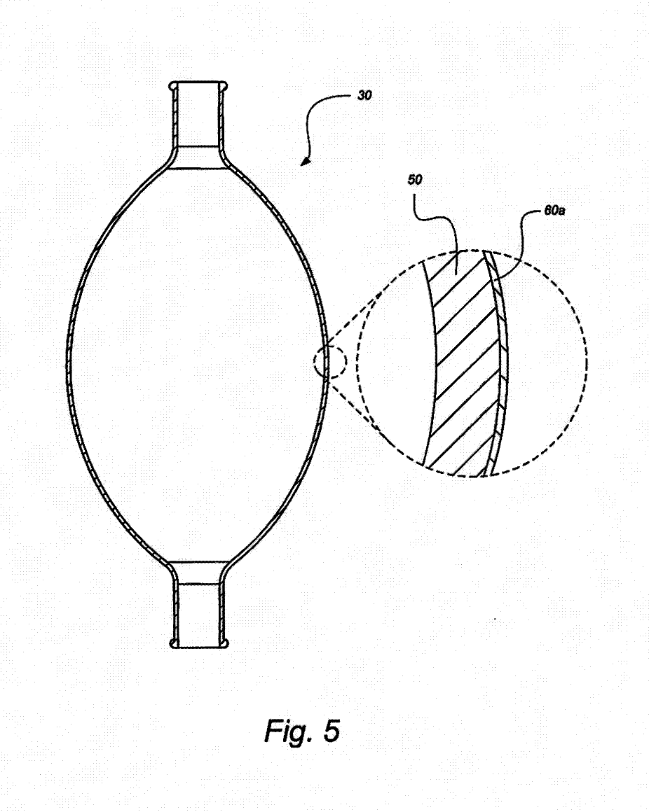

According to embodiments, and as shown in FIG. 5, an outer surface of core 50 may be provided with outer coating 60a. Outer coating 60a may be provided, for example, by a dipping process after core 50 is formed.

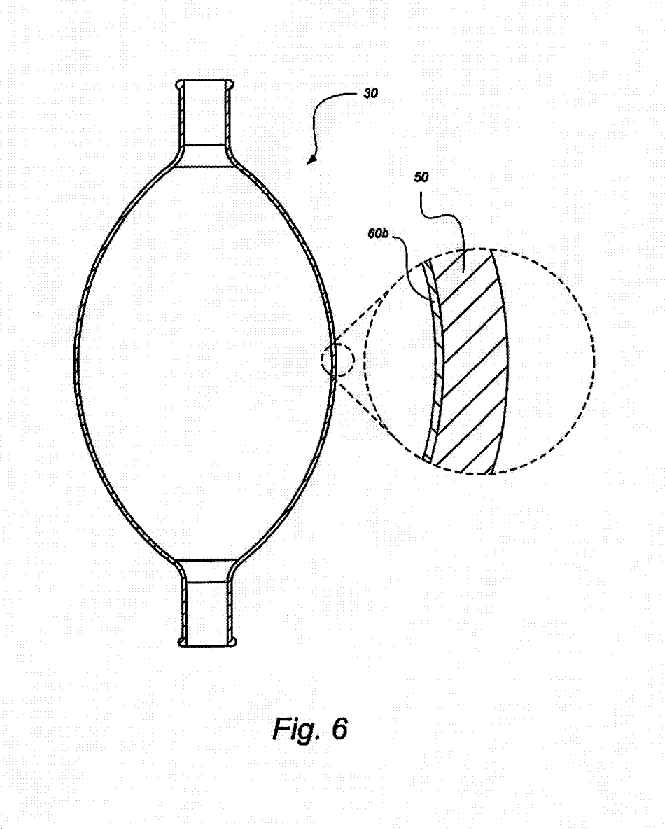

According to embodiments, and as shown in FIG. 6, an inner surface of core 50 may have inner coating 60b. Inner coating 60b may be provided, for example, by a dipping process after core 50 is formed.

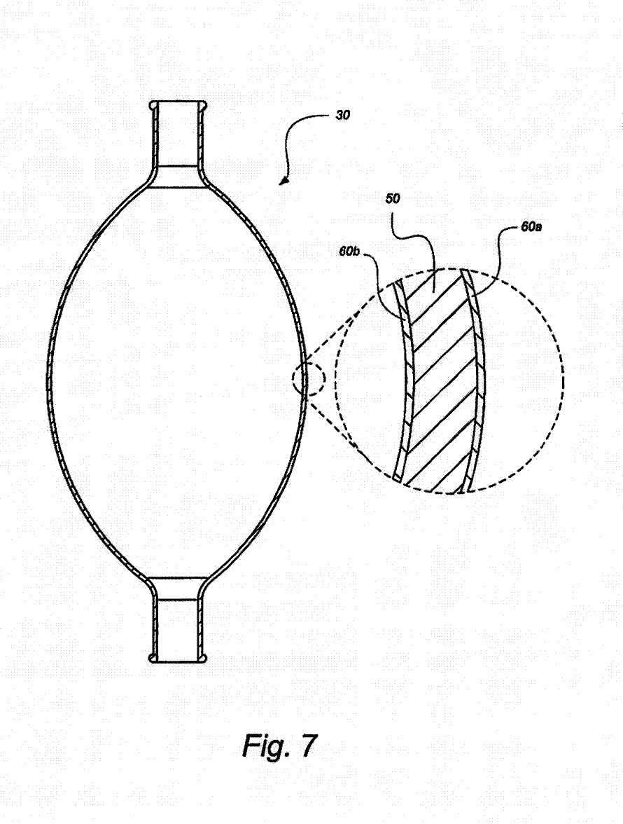

According to embodiments, and as shown in FIG. 7, an outer surface and an inner surface of core 50 may have outer coating 60a and inner coating 60b. Outer coating 60a and inner coating 60b may be provided, for example, by a dipping process after core 50 is formed.

According to embodiments, balloon 30 having layered multiple-material composition may benefit from the advantaged of each material while mitigating or minimizing the detriments of each.

According to embodiments, a method of making balloon 30 is disclosed. According to embodiments, core 50 may be molded of liquid silicone rubber (LSR) grade material, such as PDMS. Those skilled in the art will recognize various molding and extrusion processes that may facilitate formation of core 50. According to embodiments, after removal of core 50 from its mold, mandrel, or other devices, core 50 may be dipped in a second substance to form at least one of outer coating 60a and inner coating 60b. Other methods may be employed, such as spraying, coating, washing, etc.

According to embodiments, core 50 may compose a substantial portion of the total sum of balloon 30 or at least the walls thereof. For example, the thickness of core 50 may be between about 0.001″ and about 1.0″. By further example, the thickness of core 50 may be between about 0.024″ and about 0.030″. Other thicknesses may be applied based on the needs and applications of the desired product.

According to embodiments, outer coating 60a or inner coating 60b may be a thin layer, relative to the thickness of core 50. For example, outer coating 60a or inner coating 60b may a thickness of about 1% to about 99% of the thickness of core 50. By further example, outer coating 60a or inner coating 60b may a thickness of about 10% to about 20% of the thickness of core 50. Other thicknesses may be applied based on the needs and applications of the desired product. It should be noted that outer coating 60a or inner coating 60b of PDPS materials increase the stiffness of balloon 30 and reduce elongation properties thereof.

According to embodiments, balloon 30 having core 50 and at least one of outer coating 60a and inner coating 60b may have a substantially consistent surface and retain elastic material properties, increased acid resistance, a longer polymer chain (resist degradation), double bonds (resist degradation), increased hydrophobicity and less permeability (reduces ingress/egress of materials).

According to embodiments, balloon 30 may be of a single blended molding/extrusion grade material. Balloon 30, or portions thereof, may be a blend of PDPS material(s) and PDMS material(s). According to embodiments, a blended material for balloon 30 comprises PDPS materials and PDMS materials. The blended material may be at least mostly of a PDMS base. The blended material may have a PDPS material compounded in. For example, a blended material may be of between about 50% and about 99% PDMS and between about 50% and about 1% PDPS.

According to embodiments, PDMS and PDPS material blends can be compounded at the raw material level by a manufacturer. Depending on the consistency (Durometer) and batch size of the material the blends can either be, but not limited to, manual mixed using basic spatulas and beaker, roller mills or any other acceptable proprietary mixing methods. Other proprietary additives may also be compounded in to enable the material to be processed in molding/extrusion fabrication processes.

According to embodiments, balloon 30 may be loaded with additional materials. For example, active materials for therapeutic use in situ may be provided within the composition of balloon 30. Said additional materials may be at least substantially homogenously distributed throughout balloon 30. Other materials considered for blending into the silicone material are salt or silver based anti-microbial additives. Other active materials for providing therapeutic benefits are contemplated by the present disclosure.

According to embodiments, the blended material is a liquid silicone rubber (LSR) grade material. The material can be injected into a molding press.

According to embodiments, a PDPS/PDMS blend material reduces processing required to manufacture balloons that are molded and dipped in sequential steps. PDPS/PDMS blends further ensure a substantially homogenous mix of the materials and properties throughout the balloon, unlike a composite of multiple layers generated with a molded and dipped balloon.

For example, a custom blended material was created by combination of a Di-methyl base material with 15% Di-phenyl material compounded in (“LSR-9958-30”). Separate lots thereof were compared to various lots of “MED-4820 Liquid Injection Molding Silicone Elastomer,” “MED-6400 Addition Cure Silicone Dispersion,” and “MED-6600 Addition Cure Silicone Dispersion,” all by NuSil Technology (Carpinteria, Calif.). The tables below demonstrate features of the materials analyzed.

TABLE 2
(Custom Blend)

| | LSR-9958-30—Lot 1 | LSR-9958-30—Lot 2 | Average |
|---|---|---|---|
| Specific Gravity | 1.21 | 1.2 | 1.2 |
| Durometer (A-Scale) | 21 | 26 | 24 |
| Tensile Psi (Min.) | 1499 | 1916 | 1708 |
| Elongation % (Min.) | 946 | 998 | 972 |
| Tear Strength Ppi (Min.) | 286 | 322 | 304 |
| Phenyl Content % | 15 | 15 | 15 |

TABLE 3
(Off the Shelf Di-Methyl Liquid Injection Molding Blend)

| | MED-4820—Lot 1 | MED-4820—Lot 2 | Average |
|---|---|---|---|
| Specific Gravity | 1.12 | 1.12 | 1.1 |
| Durometer (A-Scale) | 23 | 22 | 23 |
| Tensile Psi (Min.) | 1254 | 886 | 1070 |
| Elongation % (Min.) | 914 | 897 | 906 |
| Tear Strength Ppi | 97 | 116 | 107 |
| (Min.) | | | |
| Phenyl Content % | 0 | 0 | 0 |

TABLE 4
(Off the Shelf Di-Methyl and Di-Phenyl Dinning Blend)

| | MED-6400 | MED-6600 |
|---|---|---|
| Specific Gravity | 1.13 | 1.17 |
| Durometer (A-Scale) | 30 | 25 |
| Tensile Psi (Min.) | 1500 | 1200 |
| Elongation % (Min.) | 775 | 750 |
| Tear Strength Ppi (Min.) | 150 | 125 |
| Phenyl Content % | 5 | 5 |

As shown, the material properties of the LSR-9958-30 are as good as or better than the lots received for MED-4820. In some areas, such as tear strength, the custom blend is magnitudes better than the MED-4820. As shown in Table 1, a typical material property of Di-Phenyl loaded blends is a lower elongation percentage. This material limitation (typically elongation % is around 750%) has been addressed without any sacrifice to tensile properties as shown in Tables 2-4 above. The standard off-the-shelf Di-Phenyl loaded blends (dipping material) contains approximately a 5% loading. Given the material property benefits achieved with the add Di-Phenyl at a 5% loading, the LSR-9958-30 blend benefited greatly from the 15% Di-Phenyl loading particularly in the area of permeability (approximately 30% percent reduction). Another mechanical property that has increased is Young's Modulus (Tensile Modulus) because tensile psi has increased while elongation has stayed relatively constant from MED-4820 to LSR-9958-30.

From the foregoing, it will be appreciated that specific embodiments of the present technology have been described herein for purposes of illustration, but that various modifications may be made without deviating from the spirit and scope of the disclosure. Aspects of the disclosure described in the context of particular embodiments may be combined or eliminated in other embodiments. Further, while advantages associated with certain embodiments of the disclosure have been described in the context of those embodiments, other embodiments may also exhibit such advantages, and not all embodiments need necessarily exhibit such advantages to fall within the scope of the disclosure. Accordingly, embodiments of the disclosure are not limited except as by the appended claims.

I/We claim:

1. An intragastric device, comprising:
an inflatable balloon comprising a substantially homogonous blend of polydiphenylsiloxane and polydimethylsiloxane material, wherein the inflatable balloon is formed by at least one of molding and extrusion.

2. The intragastric device of claim 1 wherein an amount of the polydimethylsiloxane of the inflatable balloon is at least equal to an amount of the polydiphenylsiloxane of the inflatable balloon, by weight.

3. The intragastric device of claim 1 wherein the polydiphenylsiloxane is about 15% of the inflatable balloon, by weight.

4. The intragastric device of claim 1 wherein the inflatable balloon is mostly of the polydimethylsiloxane, by weight.

5. The intragastric device of claim 1 wherein the inflatable balloon further comprises an active material.

6. The intragastric device of claim 5, wherein the active material is an anti-microbial additive.

7. The intragastric device of claim 1 wherein the inflatable balloon has a greater elongation percentage limit than a non-molded and/or non-extruded balloon consisting of a polydiphenylsiloxane and polydimethylsiloxane blended material.

8. The intragastric device of claim 1 wherein the inflatable balloon has a greater tear strength than a non-molded and/or non-extruded balloon consisting of a polydiphenylsiloxane and polydimethylsiloxane blended material.

9. The intragastric device of claim 1 wherein the inflatable balloon has a greater tear strength than a balloon formed by any manufacturing method and consisting of polydimethylsiloxane alone.

10. The intragastric device of claim 1 wherein the inflatable balloon has a lower permeability than a non-molded and/or non-extruded balloon consisting of a polydiphenylsiloxane and polydimethylsiloxane blended material.

11. The intragastric device of claim 1 wherein the inflatable balloon has a lower permeability than a balloon formed by any manufacturing method and consisting of polydimethylsiloxane alone.

12. The intragastric device of claim 1 wherein the inflatable balloon has a greater Young's Modulus than a balloon consisting of polydimethylsiloxane alone.

13. The intragastric device of claim 1 further comprising a shaft.

14. The intragastric device of claim 13, wherein the inflatable balloon further comprises two cuffs configured to receive the shaft extended through the inflatable balloon.

15. A method of producing an inflatable balloon, comprising:
providing a cavity that defines an outer shape of the balloon;
providing a mandrel defining an inner shape of the balloon; and
providing a substantially homogonous blend of polydiphenylsiloxane and polydimethylsiloxane material within the cavity and around the mandrel, whereby the balloon is defined by the space between the cavity and the mandrel.

16. The method of claim 15 wherein the balloon has a cuff at an end portion of the balloon and a middle diameter corresponding to a middle diameter of the mandrel.

17. The method of claim 16, wherein the middle diameter of the mandrel is between about 400% and about 600% larger than a diameter of the cuff.

18. The method of claim 15, further comprising:
removing the balloon from the cavity around an exterior of the balloon; and
removing the balloon from the mandrel by passing the mandrel through a cuff at an end portion of the balloon.
